(19) United States
(12) Patent Application Publication
(10) Pub. No.: US 2003/0162183 A1
(43) Pub. Date: Aug. 28, 2003

(54) Array design system and method

(76) Inventor: Robert Kincaid, Half Moon Bay, CA (US)

Correspondence Address: Agilent Technologies, Inc, Legal Department, DL429, Intellectual Property Administration, P.O. Box 7599, Loveland, CO, 80537-0599, US

(21) Appl. No.: 10/087,035

(22) Filed: Feb. 27, 2002

(51) Int. Cl. G06F 17/50; C12Q 1/68; C12M 1/34

(52) U.S. Cl. 435/6; 435/287.2; 703/1

(57) ABSTRACT

Systems and methods for array design that allow users or customers of arrays to input various selectable array design parameters that are usable by a specialized array designer or vendor for preparation of completed array designs or fabricated array chips. The methods comprise selecting at least one array design parameter by an array customer, transferring or providing the selected array parameter to an array vendor, determining, by the vendor, one or more additional array design parameters, and completing an array design or designs according to the customer selected array parameter and vendor provided array parameter. The systems comprise one or more data processors having stored programming configured to allow an array customer to enter or input selectable array parameters, view selected array parameters and, if desired, revise the selected array parameters.

### BACKGROUND OF THE INVENTION

Arrays of biomolecules and other molecules of interest are widely used as tools in high throughput technologies, including gene expression analysis, genotyping, nucleic acid analysis, nucleic acid sequencing, mutation analysis, protein and peptide analysis, and screening of potential drugs. Biomolecule arrays also find increasing use as combinatorial chemistry platforms for drug development and manufacturing.

One common type of array utilizes a plurality of nucleic acid probes of selected sequences that are arranged in a selected pattern on a substrate surface. The probe sequences and arrangement of probes in the array may be configured for gene-based analysis such as gene expression analysis, sequence analysis or characterization of other genomic information by a match/mismatch probe strategy. One or more labeled nucleic acids are applied to the array under appropriate conditions, and the arrays are scanned or otherwise characterized to determine the location of labeled nucleotides bound to probes in the array. The desired genomic information may be obtained from the probe sequences and probe locations associated with the bound, labeled nucleic acids.

The design of arrays typically involves complex and specialized computational techniques. In particular, the computational aspects associated with probe selection and sequence curation for nucleic acid arrays are usually too technical and burdensome for the typical user of such arrays. Further, the specialized hardware and software used in probe selection and sequence curation for nucleic acid array involve substantial cost. The design of arrays has thus been problematic and commercially unsupportable for the average array user. For these reasons, array design and manufacture has largely been left to specialized commercial suppliers of arrays. The sophisticated array design processes used by such commercial suppliers is generally not adaptable to individual needs or customization in array design, and the end-users of arrays typically have little or no input in the array design process.

The level of sophistication of array users has increased as array technology has progressed, and the amount of information usable in array design has increased and become more widely available. Commercial array users are increasingly interested in becoming directly involved in various aspects of the array design process. No systems or methods exist, however, that permit array users to selectively input array design parameters for use by commercial array designers and manufacturers.

There is accordingly a need for an array design system and method that simplifies array design, that allows selective input of array design parameters by commercial array users, that can isolate such users from complex computational aspects of array design, and which allows quick and easy sharing of array design parameter information between commercial array users and array designers and manufacturers. The present invention satisfies these needs, as well as others, and overcomes the deficiencies found in the background art.

Relevant Literature

U.S. patent documents of interest include U.S. Pat. Nos. 5,593,839, 5,856,101, 6,188,783, 6,251,588 and 6,229,911.

### SUMMARY OF THE INVENTION

The invention provides systems and methods for array design that allow users or customers of arrays to input various selectable array design parameters that are usable by a specialized array designer or vendor for preparation of completed array designs or fabricated array chips. The systems and methods of the invention permit decoupling of computation-intensive aspects of array design from simpler aspects of the design process. The level of array parameter input by customers can be varied according to the interests and sophistication level of the individual customers.

The subject methods comprise, in general terms, selecting at least one array design parameter by an array customer, transferring or providing the selected array parameter to an array vendor, determining, by the vendor, one or more additional array design parameters, and completing an array design or designs according to the customer selected array parameter(s) and vendor provided array parameter(s). Completion of the array design may be carried out by the customer or the vendor. The methods may further comprise fabrication of the array according to the completed array design. The array fabrication may comprise in situ synthesis of probes on an array substrate surface according to a completed array design.

By way of example, and not of limitation, the array parameters selected by the customer may be gene-based, for design of nucleic acid probe arrays. The customer selectable array parameters may comprise layout parameters, probe parameters, control probe parameters, or other array design parameters. The array parameters provided by the vendor may comprise any parameters not provided by the customer that allow completion of an array design. The completed array design may be delivered to the customer for use in array fabrication by the customer, or the vendor may fabricate the array according to the completed array design and deliver the array to the customer.

In certain embodiments, the invention provides methods for gene-based design of an in-situ array, which comprise selecting, by a customer, at least one gene of interest, selecting, by the customer, at least one array design parameter for the gene of interest, providing the customer selected array design parameter to a vendor, providing, by the vendor, at least one additional array design parameter for the gene of interest, and completing at least one array design according to the customer-selected array design parameters and the vendor provided array design parameters. The method may additionally comprise synthesizing nucleic acid probes on a substrate surface, according to the completed array design to provide the in-situ array.

In other embodiments, the invention provides methods for gene-based array design comprising selecting, by a customer, at least one gene of interest, selecting, by the customer, at least one probe parameter for the gene of interest, selecting, by the customer, at least one array layout parameter for the gene of interest, curating, by a vendor, sequence information for the gene of interest, and selecting, by the vendor, a plurality of nucleic acid probes for the gene of interest.

The systems of the invention comprise, in general terms, one or more data processors having stored programming configured to allow an array customer to enter or input selectable array parameters, view selected array parameters and, if desired, revise the selected array parameters. The selectable array parameters may be gene-specific for nucleic acid arrays. In certain embodiments, the system may comprise a single, personal computer or other data processor used by a customer. The stored programming may be configured to allow the array customer to select which array parameters that the customer wishes an array vendor to provide. In other embodiments, one or more customer client computers may be networked to one or more array vendor server computers via computer network such as the Internet. The stored programming may operate in a stand-alone manner on a customer's computer or computers, or as a web-based application accessible to the customer computer. The programming provides a visual user interface on the customer client computer for parameter selection and input, review of selected parameters, and revision of selected parameters by customers. The visual interface may provide a display of an array layout based on the customer parameter selections, which may be reviewed and revised by additional customer parameter selection by the customer, or modification of previous array parameter selections.

The systems may further comprise stored programming configured to output customer-selected array parameters to a commercial array vendor, and to allow the vendor to input additional array parameters not selected by the customer. The systems may further comprise one or more databases of array design information, accessible by customer client computers and/or vendor computers, that contain information usable in array parameter selection by customers and or vendors.

The invention also provides computer readable media with stored programming configured to allow an array customer to input one or more selectable array parameters, and to generate a visual user interface which displays an array layout or other aspect(s) of any array design. The computer readable media may further comprise stored programming configured to allow an array vendor to utilize array parameters selected by a customer for preparing a completed array design. The programming may provide a visual user interface for parameter selection that permits selective inputting of parameters as well as selective deferring of parameter selections to a vendor. The programming may be configured to allow customer parameter selection on a gene-specific basis.

The invention also provides kits for array design which may comprise a computer readable medium with stored programming thereon configured for inputting of one or more selectable array parameters by a user, together with printed instructions for the selection of array design parameters. The kits may further comprise devices and materials for isolation and/or characterization of nucleic acids or other molecules of interest such as PCR (polymerase chain reaction) related items, as well as printed instructions associated with the isolation and/or characterization of the molecules of interest.

The invention is well suited for use in gene-centric design of custom “in situ” oligomer and long oligo arrays wherein nucleic acid oligos are synthesized directly on an array substrate. Such arrays are typically synthesized in relatively small numbers, and probe design is critical for selection of good probes. The invention may also be used in the design of cDNA arrays wherein clones of genes of interest are prepared in advance and applied to arrays by pin-based spotters, as well as spotted oligo arrays wherein pre-synthesized short nucleotide probes of known sequence are spotted on arrays.

### BRIEF DESCRIPTION OF THE DRAWINGS

FIG. 1 is a flow chart illustrating an array design method in accordance with the invention.

FIG. 2 is a functional block diagram illustrating one embodiment of an array design system in accordance with the invention.

FIG. 3 is a flow chart illustrating an embodiment of an array design method usable with the array design system of FIG. 2.

### DETAILED DESCRIPTION OF THE INVENTION

Disclosed herein are systems and methods for array design that permit array customers or users to input selected parameters associated with array design while allowing other design parameters to be selected or determined by a specialized array designer or manufacturer. The invention allows array customers a wide range of input into the array design process according to the interest and sophistication level of individual customers, while also allowing customers to defer selection of difficult, computationally intensive design considerations to array design specialists.

Before the subject invention is described further, it should be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

It should also be noted that as used herein and in the appended claims, the singular forms “a”, “and”, and “the” include plural referents unless the context clearly dictates otherwise. Thus, for example, reference to “a probe” includes a plurality of such probes, and reference to “the array” includes reference to one or more arrays and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. The dates of publication provided may be different from the actual publication dates, which may need to be independently confirmed. All publications mentioned herein are incorporated herein by reference to disclose and describe the methods, systems or other subject matter in connection with which the publications are cited.

Any definitions herein are provided for reason of clarity, and should not be considered as limiting. The technical and scientific terms used herein are intended to have the same meaning as commonly understood by one of ordinary skill in the art to which the invention pertains.

As used herein, “array”, “microarray” and grammatical equivalents thereof refer to any device wherein molecules are arranged on a substrate surface in a selected pattern. Molecules used in an array may comprise, by way of example and not of limitation, nucleic acids such as monomeric, oligomeric or polymeric DNA or RNA, peptides, proteins, or other organic or biological molecules of interest.

As used herein “in situ array and grammatical equivalents thereof refer to array devices having molecules that are grown or synthesized on a substrate surface.

As used herein, “parameter” and grammatical equivalents thereof refers to any data, value, feature, or information that can be used in the designing and fabrication of an array. Parameters usable in the design of arrays include, by way of example and not of limitation, probe parameters such as the number of probes per array, probe length(s), probe sequences, the number of probes per gene versus replicate probes, control probe parameters such as the number of control probes per gene, control probe sequences (from sets of standard sequences), the inclusion or exclusion of deletion controls, array layout parameters such as general layout patterns, the number of features per array, probe densities, spacing between probe spots, tiling considerations such as probe groupings, gene-based positioning of probes on the array, selection of probe pair sets (arrangement of reference and partner probes), control probe layouts, and parameters associated with array fabrication such as the number and types of pins used for depositing probe spots, the number of pin cleaning cycles, or mask design considerations for photolithographic array fabrication techniques.

As used herein, “customer” and grammatical equivalents thereof refers to any individual, person, or entity that may wish to obtain an array design using the systems and methods of the invention. Array “customers” in many situations may comprise commercial array users having a need for completed array designs.

As used herein, “vendor” and grammatical equivalents thereof means any individual, person, or entity that is capable of creating or constructing a completed array design according to customer-selected array parameters provided by the systems and methods of the invention. Array “vendors” in many instances may comprise commercial array designers or fabricators that have access to sophisticated array fabrication tools.

As used herein, “nucleic acid” and grammatical equivalents thereof means a nucleotide monomer, oligomer or polymer.

As used herein, “nucleotide” and grammatical equivalents thereof refers to a nucleic acid that includes a phosphate group, a 5 carbon sugar and a nitrogen containing base, as well as analogs of such subunits. “Nucleotide” includes deoxyribonucleotides and ribonucleotides.

As used herein, “ribonucleic acid”, “RNA” and grammatical equivalents thereof means a monomer, polymer or oligomer composed of ribonucleotides.

As used herein, “deoxyribonucleic acid”, “DNA” and grammatical equivalents thereof means a polymer composed of deoxyribonucleotides.

As used herein, “oligonucleotide”, “oligo”, and grammatical equivalents thereof denote single stranded nucleotide multimers of from about 10 to up to about 100 nucleotides in length.

As used herein, “polynucleotide” and grammatical equivalents thereof refers to a single or double stranded polymer composed of nucleotide monomers of generally greater than 100 nucleotides in length and up to about 8,000 or more nucleotides in length. Polynucleotides include single or multiple stranded configurations, where one or more of the strands may or may not be completely aligned with another.

As used herein, “peptide” and grammatical equivalents thereof refers to any compound produced by amide formation between a carboxyl group of one amino acid and an amino group of another group.

As used herein, “oligopeptide” and grammatical equivalents thereof refers to peptides with fewer than about 10 to 20 residues, i.e. amino acid monomeric units.

As used herein, “polypeptide” and grammatical equivalents thereof refers to peptides with more than 10 to 20 residues.

As used herein, “protein” and grammatical equivalents thereof refers to polypeptides of specific sequence of more than about 50 residues.

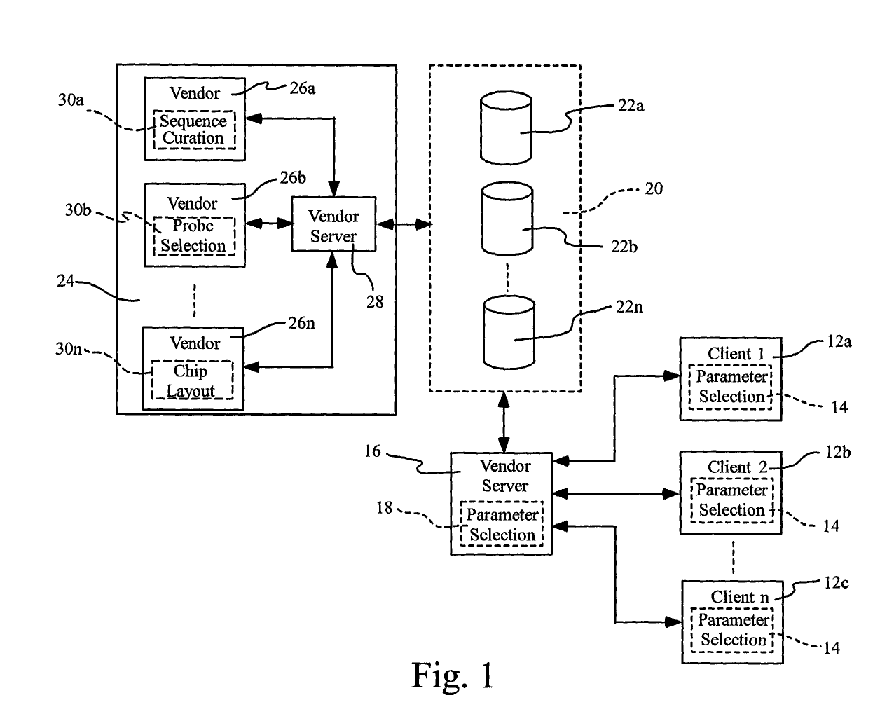

With the above in mind, reference is made more specifically to the drawings in which, for illustrative purposes, show the present invention embodied in systems and methods in FIG. 1 through FIG. 3. It will be appreciated that the systems may vary as to configuration and as to details of the parts, and that the methods may vary as to detail and the order of the events or acts, without departing from the basic concepts as disclosed herein. The invention is described primarily in terms of use with nucleic acid microarrays for gene-based analysis. The invention may be used, however, in the design of arrays of any molecules of interest, including oligomeric and polymeric DNA and RNA, peptides, proteins, and the like. The invention is also described in terms of customers and vendors of arrays, and it should be understood that the users of the invention need not necessarily be commercial customers and vendors of array products, but may be any array users and designers. It should also be apparent to those skilled in the art that various functional components of the invention as described herein may share the same logic and be implemented within the same circuit, or in different circuit configurations.

Systems

The invention may be embodied in a variety of stand-alone or networked computer or data processor systems. In some embodiments, the subject array design methods may be carried out in association with a stand alone program operating on a single computer or data processing device, while in other embodiments a portion or all of the array design methods may be carried out in association with a web-based application residing on one or more server computers that are accessed by client computer via network interface.

Referring to FIG. 1, there is shown one embodiment of an array design system 10. The system 10 includes a plurality of client computers 12a, 12b, 12n, each of which may comprise a standard computer such as a minicomputer, a microcomputer, a UNIX® machine, mainframe machine, personal computer (PC) such as INTEL®, APPLE®, or SUN® based processing computer or clone thereof, or other appropriate computer. Client computers 12a, 12b, 12n may also include typical computer components (not shown), such as a motherboard, central processing unit (CPU), memory in the form of random access memory (RAM), hard disk drive, display adapter, other storage media such as diskette drive, CD-ROM, flash-ROM, tape drive, PCMCIA cards and/or other removable media, a monitor, keyboard, mouse and/or other user interface, a modem, network interface card (NIC), and/or other conventional input/output devices. In many embodiments, client computers 12a, 12b, 12n comprise conventional desktop or “tower” machines, but can alternatively comprise portable or “laptop” computers, handheld personal digital assistants (PDAs), cellular phones capable of browsing Web pages, “dumb terminals” capable of browsing Web pages, internet terminals capable of browsing Web pages such as WEBTV®, or other Web browsing or network enabled devices.

Each client computer 12a, 12b, 12n may comprise, loaded in its memory, an operating system (not shown) such as UNIX®, WINDOWS® 98, WINDOWS® ME, WINDOWS® 2000 or the like. Each client computer 12a, 12b, 12n may further have loaded in memory a Web Browser program (not shown) such as NETSCAPE NAVIGATOR®, INTERNET EXPLORER®, AOL®, or like browsing software for client computers. In accordance with the invention, client computers 12a, 12b, 12n may each comprise array parameter selection programming 14 stored in memory that allows users of client computers 12a, 12b, 12n to selectively input various parameters associated with array design on a gene-specific basis, as described further below. Programming 14 may be the form of electronically, optically, or magnetically stored code or other form of computer readable stored code, that is loaded in the RAM or other memory of client computers 12a-12n. In the embodiment shown in FIG. 1, each client computer 12a-12n represents a computer used by an individual array customer for the selection and inputting of parameters used in the design of an array.

The system 10 also comprises one or more vendor servers 16, which may be any standard data processing device or computer, including a minicomputer, a microcomputer, a UNIX® machine, a mainframe machine, a personal computer (PC) such as INTEL® based processing computer or clone thereof, an APPLE® computer or clone thereof or, a SUN® workstation, or other appropriate computer. Vendor server 16 may include conventional computer components (not shown) such as a motherboard, central processing unit (CPU), random access memory (RAM), hard disk drive, display adapter, other storage media such as diskette drive, CD-ROM, flash-ROM, tape drive, PCMCIA cards and/or other removable media, a monitor, keyboard, mouse and/or other user interface means, a modem, network interface card (NIC), and/or other conventional input/output devices.

Vendor server 16 has stored in its memory a server operating system (not shown) such as UNIX®, WINDOWS® NT, NOVELL®, SOLARIS®, or other server operating system. Vendor server 16 also has loaded in its memory web server software (also not shown) such as NETSCAPE®, INTERNET INFORMATION SERVER™ (IIS), or other appropriate web server software loaded for handling HTTP (hypertext transfer protocol) or Web page requests. Vendor server 16 may also comprise stored array parameter selection programming 18 that allows users of client computers 12a, 12b, 12n to selectively input various parameters associated with array design using array parameter selection programming 18 as described further below. Programming 18 may be the form of electronically, optically, or magnetically stored code or other form of computer readable stored code, that is loaded in the RAM or other memory of vendor server 16.

Client computers 12a, 12b, 12n are operatively coupled to vendor server 16 for communication with vendor server 16 via the Internet (not shown) or other computer network using DSL (digital subscriber line), telephone connection with a modem and telephone line via an internet service provider (ISP), wireless connection, satellite connection, infrared connection, or other means for establishing a connection to the Internet. Vendor server 16 may be connected to the Internet by a fast data connection such as T1, T3, multiple T1, multiple T3, or other data connection. Client computers 12a, 12b, 12n and vendor server 16 communicate via the Internet or other network connection using the TCP/IP (transfer control protocol/internet protocol) or other network communication protocol.

The system 10 includes a data bank 20 that may comprise one or more individual databases 22a, 22b, 22n which are operatively coupled to vendor server 16. Vendor server 16, in this regard, may include stored database management programming such as SQL®, DB2® or like programming capable of retrieving and storing information in association with databases 22a, 22b, 22n. Vendor server 16 alternatively may be operatively coupled to databases 22a, 22b, 22n through one or more database servers (not shown) that are capable of accessing information from databases 22a, 22b, 22n.

Databases 22a-22n may include, inter alia, stored information usable by commercial array clients for the selection and inputting of array design parameters according to programming 14, 18. Data bases 22a, 22b, 22n are also configured to store selected array design parameters from commercial array clients for subsequent use, by a commercial array vendor, for preparing completed array designs and/or fabrication of arrays according to selected array parameters. Databases 22a-22n may comprise, in whole or in part, proprietary databases created by a commercial array vendor or vendors for use by the vendor's array customers. One or more of databases 22a-22n may alternatively comprise proprietary databases of individual array clients that are used by individual array clients for selection of array parameters.

Databases 22a-22n may additionally comprise databases of genomic information that are accessible to the general public via the Internet. Such databases may include molecular, genetic, organism-based, gene expression, bibliographic, or other type of genomic information usable for selection of array design parameters. Public, Internet-accessible data bases that provide information usable in selection of array design parameters include, by way of example, European Molecular Biology Laboratory Nucleotide Sequence Data Library (EMBL), http://www.embl-heidelberg.de/, DNA Database of Japan (DDBJ), http://www.ddbj.nig.ac jp/, Genbank, http://www.ncbi.nlm.nih.gov/Genbank/GenbankSearch.html, Swiss-Prot., http://www.expasy.ch/sprot/sprot-top.html, Genome Database (GDB), http://gdbwww.gdb.org, Online Mendelian Inheritance in Man (OMIM), http://www3.ncbi.nlm.nih.gov/Omim/, Cellular Response Database, http://LHI5.umbc.edu/crd, dbEST, http://www.ncbi.nlm.nih.gov/dbEST/index.html, GeneCards, http://bioinformatics.weizmann.ac.il/cards/, Globin Gene Server, http://globin. cse.psu.edu, Human Developmental Anatomy, http://www.ana.ed.ac.uk/anatomy/database/humat/, Kidney Development Database, http://www.ana.ed.ac.uk/anatomy/database/kidbase/kidhome.html, Merck Gene Index, http://www.merck.com/mrl/merck_gene_index.2.html, and Tooth Gene Expression Database, http://bite-it.helsinki.fi/. Proprietary databases are also accessible via the Internet for a fee or on a subscription basis, such as Incyte's LIFESEQ®, http://www.incyte.com/sequence/index.shtml, and DOUBLETWIST™, https://genomezone.doubletwist.com/. Various other public-accessible databases are known to those skilled in the art and may be used for databases 22a, 22b, 22n as well.

Stored information in databases 22a, 22b, 22n may comprise, for example, information usable by array clients for selection of probe parameters, such as the number of probes per array, probe length(s), the number of probes per gene versus replicate probes, and other probe parameters. Where sufficient information is available for particular genes of interest, the stored information for probe parameters may also comprise established probe sequence information for selection of probe sequencing. Sophisticated array clients may utilize genomic information in databases 22a, 22b, 22n for selection of sequence-related probe parameters through use of accession numbers or database search algorithms such as FASTA, BLAST, the Smith-Waterman algorithm, or other sequence search algorithm. It is contemplated that many commercial array customers would defer selection of probe sequence parameters to specialized array vendors, as described more fully below.

The stored information for array parameter selection in databases 22a, 22b, 22n may also comprise information related to selection of control probes, including the number of control probes, control probe sequences (from sets of standard sequences), the inclusion or exclusion of deletion controls, or other control probe considerations. Databases 22a, 22b, 22n may additionally comprise information usable for selection of array layout parameters such as general layout patterns, the number of features per array, probe densities, spacing between probe spots, tiling considerations such as probe groupings, gene-based positioning of probes on the array, selection of probe pair sets (arrangement of reference and partner probes), control probe layouts, and other layout considerations.

Information in databases 22a, 22b, 22n may still further comprise information usable for array fabrication. Sophisticated commercial array users may wish to select parameters such as, for example, the number and types of pins, the number of pin cleaning cycles, or other parameters associated with printer configuration during array fabrication. Again, it is contemplated that many commercial array users will not wish involvement in the actual details of array fabrication, and will leave selection of such considerations to specialized array vendors.

The information in databases 22a, 22b, 22n may be configured in a variety of arrangements known to those skilled in the art. The databases 22a, 22b, 22n may comprise relational databases wherein probe parameter selection information, control probe parameter selection information, and layout parameter selection information are arranged as tables of records stored in computer-readable media. One exemplary database structure that is usable for design of arrays is provided in U.S. Pat. No. 6,188,783. Bioinformatics database structures and methods are also described in U.S. Pat. No. 6,229,911.

The system 10 as shown in FIG. 1 includes a vendor local area network or LAN 24 that comprises one or more vendor computers 26a, 26b, 26n operatively coupled to a second vendor server 28 within LAN 24. Vendor computers 26a-26n may comprise any of the computer or data process devices described above for client computers 12a-12n, with conventional operating system and browser software as described above, and vendor LAN server 28 may comprise a computer configured in a manner similar to vendor server 16 described above. Vendor LAN server 28 is operatively coupled to databases 22a, 22b, 22n via the Internet. A firewall (not shown) may be used in association with LAN 24 for filtering inbound and outbound traffic. Vendor LAN server 28 may include web server software (not shown) for handling HTTP page requests from vendor computers 26a-26n, as well as software (also not shown) for storing and retrieving information in association with databases 22a-22n.

Vendor computers 26a, 26b, 26n respectively include stored programming 30a, 30b, 30n that is configured to allow users of vendor computers 26a-26n to input customer selected parameters for array design, to select and input any array design parameters not provided by customers, and to construct or create completed array designs according to customer-supplied and vendor-supplied design parameters. Vendor computer 26a is shown, for exemplary purposes, as having stored programming 30a usable for sequence curation aspects of array design, while vendor computer 26b includes programming 30b specific for probe selection, and vendor computer 26n includes stored programming 30c for array chip layout design.

The array design system 10 of FIG. 1 provides only one embodiment of the array design systems of the invention, and numerous variations on the system 10 will suggest themselves to those skilled in the art upon review of this disclosure. In simpler embodiments, the system of the invention may comprise a single computer with stored programming capable of carrying out the array parameter selection methods of the invention, and with a stored database of usable array design parameters. In some embodiments, the system of the invention may reside in a single client computer 12a and a single vendor computer 30a, with client computer programming 14 configured to allow customers to select and input array parameters, and with vendor computer programming 30a being configured to allow a vendor to input any array parameters not provided by the customer as required for developing a completed array design or designs. The selected customer array parameters may be transferred from client computer 12a to vendor computer 26a by physical transfer of a CD, floppy disk, or like medium, or may be transferred computer network or other interface connection.

The arrangement of vendor LAN 24 as shown in FIG. 1 is also only exemplary and may be varied. A separate vendor computer 26a-26n is shown for each of some of the more computationally intensive aspects of array design. In other embodiments, all aspects of programming 30a-30n may be stored in the memory of a single vendor computer, which may access databases 22a-22n through vendor server 28, through an external server unrelated to the vendor, or which may be isolated from the Internet. In still other embodiments, vendor computers 26a-26n may comprise individual, independent computers that are not part of a vendor LAN.

For reason of clarity, databases 22a-22n are shown in FIG. 1 as being within a single databank 20 that is accessible by both client computers 12a-12n and vendor computers 26a-26n via the Internet. This arrangement of databases 22a-22n may vary in different embodiments of the invention. In certain instances, for example, an individual customer may make array design parameter selections based on array parameter information stored directly on client computers 12a-12n. Client computers 12a-12n may be located within customer corporate LANs and utilize proprietary databases accessible only to that particular customer. Such customer proprietary databases may be firewall protected within a corporate LAN of the individual customer and accessed by an internal database server of the customer.

Similarly, an array vendor may maintain one or more proprietary databases within vendor LAN 24 for exclusive user by the vendor, wherein array design parameter information is stored that is not accessible to array customers at client computers 12a-12n. Databases 22a-22n may also comprise one or more databases such as Genbank or LIFESEQ®, as noted above, that are accessible by the public via the Internet. Client computers 12a-12n and vendor computers 26a-26n may access such public databases via third party servers (not shown), rather than via vendor server 16 or vendor LAN server 28 as shown in FIG. 1.

Databases 22a-22n may be vendor proprietary databases wherein customer access to the databases is be made on a subscription or fee basis. Thus, the customer users of client machines 12a-12n may pay a monthly or annual subscription fee, or pay fees on a per-search basis, for use of databases 12a-12n to access array design information. Access to databases 22a-22n by customers via vendor server 16 may be secure and subject to authorization or authentication of customer users prior to access. Numerous other database arrangements for the system 10 will suggest themselves to those skilled in the art, and are considered to be within the scope of this disclosure.

Parameter selection programming 14 may be provided by a vendor to customers on computer readable media such as a CD for installation on client computers 12a-12n. Alternatively, parameter selection programming 14 may be downloaded to client computers 12a-12n from vendor server 16 or other server (not shown) via the Internet. Parameter selection programming 14 may be made available to array customers on a cost basis on a subscription basis or one-time fee basis, and may be periodically upgraded by the vendor according to advances in array design technology. Where parameter selection is carried out via web based programming 18, Internet access to programming 18 on vendor server 16 may be subscription-based and subject to customer authentication prior to access. Web-based programming 18 may also comprise extension application associated with third party servers (not shown) that provides access to programming 18 on vendor server 16 via the third party servers.

Methodology

The invention provides methods that allow the customers or end-users of arrays to participate in the array design process together with a commercial array vendor. The methods comprise selecting array design parameters by a customer, displaying and reviewing the selected parameters by the customer and, if desired, revising the parameter selections prior to transmitting or outputting the selected parameters to an array vendor or specialist for completion of the array design. The methods may further comprise selecting or providing, by a vendor, any array design parameters not provided by the customer, and creating a complete array design from the selected array design parameters.

The methods of the invention permit array customers to participate in the array design process to the extent desired by individual array customers according to the interests and sophistication level of the array customers. Array customers may provide most or substantially all of the array design parameters necessary for design of an array, such that the array vendor need only finalize an array design according to the customer's selected parameters by providing one or more additional design parameters. Alternatively, the customers may provide only a single array parameter, with most of the array parameter selection being left to the array vendor.

Particularly, the inventive methods provide for decoupling the more difficult computational aspects of array design, such as sequence curation and selection of probe sequences, from simpler aspects of the array design process such as array layout considerations. Array customers thus can remain isolated from the burden of sequence curation and sequence selection computations, but can participate in selection of design parameters for array layout and other array design considerations. Parameter selection by users may be on a gene-specific, rather than probe-specific basis, to facilitate customer selection of array design parameters. Parameter selection may be carried out on site by customers, with the customers being able to view and visually adjust array layout according to selected parameters. Once the customer has reviewed and finalized parameter selection, the customer's parameter selections are transferred to the vendor for completion of the array design.

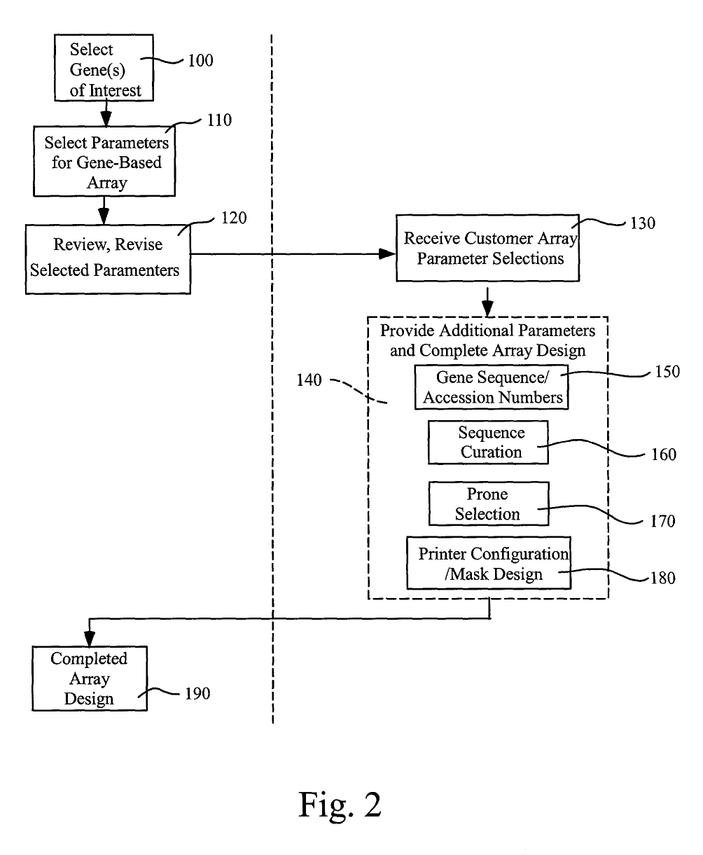

The methods of the invention will be more fully understood by reference to FIG. 2, wherein a flow chart illustrates one embodiment of the subject methods. The events shown to the left of the dashed line in FIG. 2 comprise events that are typically carried out by, or in association with, an array customer, while the events to the right of the dashed line comprise events that are typically, but not necessarily, carried out by an array vendor.

At event 100, a commercial array user or customer selects one or more genes of interest for study using an array or arrays of nucleic acids. Gene selection will depend on individual customer interests. The basis for customer gene selection may, for example, involve gene expression analysis for identification of novel genes, identification of potential drug targets, identification of markers for pathological processes, or elucidation of molecular events associated with drug treatment or effects of disease.

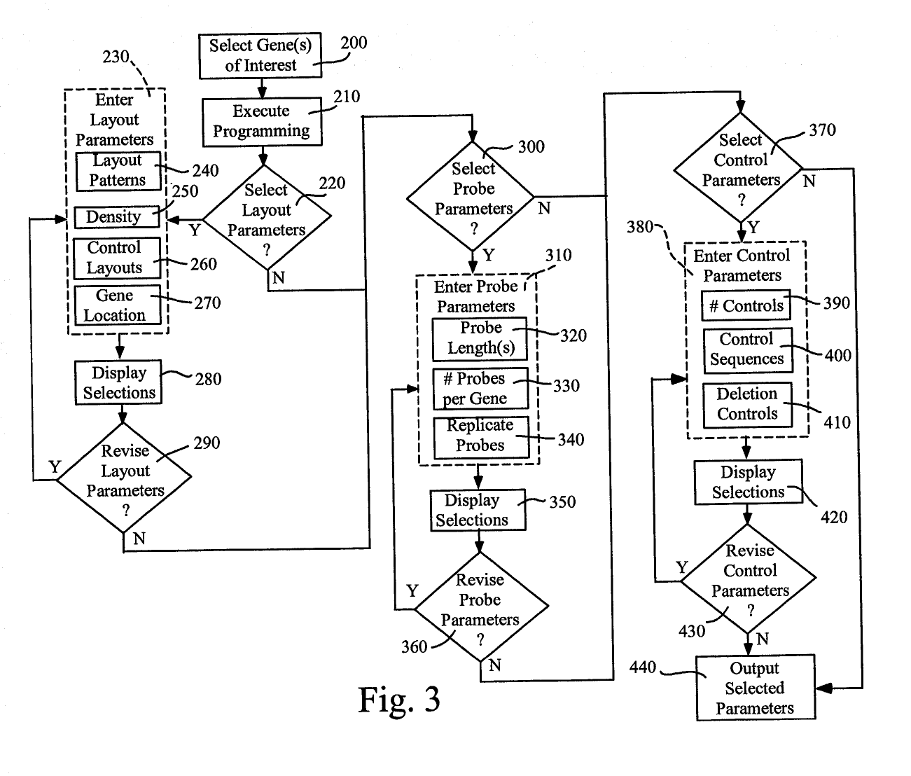

At event 110, the array customer selects one or more array design parameters usable for creating an array design. Parameter selection is carried out in association with software or programming operating on one or more computers, as described further below with reference to FIG. 3 and FIG. 1. Customer-selected parameters may include any parameters usable for array design. Customer selected parameters may include, inter alia, gene-based layout parameters such as the number and types of gene-based features per array, array size, probe spot densities, spacing between probe spots, probe groupings, gene-based positioning of probes on the array, arrangement of reference and partner probes, and arrangement of control probes. Customers may also select probe-related parameters such as probe length(s), the number of probes per gene versus replicate probes, and control probe parameters.

At event 120, the customer reviews the array design parameters selected in event 110 and, if desired, revises the selected parameters of event 110. Event 120 is carried out in association with programming operations as described below, such that a visual user interface is provided to the customer for visualization of array layout according to parameters selected in event 110. The customer may, upon visual review of the array layout in event 120, revise or re-select one or more parameters, or select one or more additional parameters.

At event 130, the customer-selected array design parameters of events 110 and 120 are transferred or otherwise provided by the customer to a commercial array vendor, and the vendor receives the customer-selected parameters.

At event 140, the array vendor completes the array design process by providing any additional design parameters needed for completion of the array design that were not provided by the customer in events 110 and 120. Array design parameters that are selected or provided by the vendor will typically be associated with computationally complex aspects of array design, i.e., nucleic acid probe sequencing parameters. Event 140 accordingly includes sub-event 150 wherein sequence information for probe selection is obtained. Sequencing information may involve database searching using accession numbers for specific sequences, or use of database search algorithms such as FASTA or BLAST to obtain raw sequence data for the customer-selected genes of interest from event 100. The FASTA and BLAST algorithms, which are well known in the art, are approximate heuristic algorithms used to compute suboptimal pairwise similarity comparisons. Dynamic programming is used to compute a series of subsequence alignments that are combined to approximate a larger sequence alignment and global similarity score. (See. e.g., http://www.nbrf.georgetown.edu/pirwww/search/fasta.html and http://www.ncbi.nlm.nih.gov/BLAST/).

Event 140 also includes sub-event 160 wherein sequence curation is carried out. Sequence curation typically involves checking the raw sequences from event 150 for errors such as incorrect sequences and incorrect 5′-3′ ordering of sequences. Sequence curation 160 may also include removal of commonly repeated subsequences such as ALU repeats and the like which would give rise to non-specific probes, and removal of any artifacts associated with sequence assembly, such as residual vector sequences. Various other methods of preparing sequences for probe selection will suggest themselves to those skilled in the art, and are considered to be within the scope of the invention.

In sub-event 170, probe selection is carried out based on the sequence information obtained in sub-events 150 and 160. This event involves determining the number of unique nucleic acid oligomers or oligos that will effectively sample the entire length of a nucleotide sequence that is hybridizable with a target sequence of a gene of interest. One or more parameters, that are independently predictive of the ability of each nucleic acid oligomer to hybridize to the target sequence, may be used to develop subsets of oligomers based on the parameters. Oligomers from the subsets can then be identified that are clustered along specific regions of the sequence that is hybridizable with the target sequence. Probes thus selected can be laid out in the user-selected layout patterns provided in events 110 and 120. An exemplary method of probe selection is disclosed in U.S. Pat. No. 6,251,588, the disclosure of which is incorporated herein by reference.

The providing of array parameters by the vendor in event 140 may additionally comprise sub-event 180, wherein array parameters associated with array fabrication processes may be selected. Array fabrication parameters may include, for example, the number and types of pins used for depositing probe spots, the number of pin cleaning cycles, or other parameters associated with robotic, inkjet deposition of probe spots. Array fabrication parameters may also comprise mask design or other photolithographic considerations associated with photolithographic array fabrication.

Following the completion of the array design by the vendor in event 140 and sub-events 150-180, the completed array design is delivered or otherwise provided to the customer in event 190. The vendor, third party or customer may then carry out array fabrication according to the completed array design.

Numerous variations on the method embodiment of FIG. 1 are possible. For example, probe sequence selection in sub-event 170 may alternatively be carried out by the customer in events 110 and 120 in cases where the customer has sufficient information available for probe sequence selection or is otherwise capable of obtaining the necessary sequence information for probe selection by genomic database mining and sequence curation. Similarly, sophisticated array customers may wish to select array fabrication parameters in events 110 and 120, rather than leave selection of such parameters to the array vendor. In some embodiments, event 140 may comprise completing of the array design by the vendor using array parameters selected entirely by the customer. In still other embodiments, customers may select only one or a few basic array layout parameters, and leave the bulk of the parameter selection process for the vendor to carry out in event 140. Various other modifications of the inventive methods will suggest themselves to those skilled in the art, and are considered to be within the scope of the invention.

Referring now to FIG. 3, the methods of the invention using the array design system 10 of FIG. 1 are described. As noted above, customer parameter selection is carried out using programming, which may operate in a stand-alone manner on a customer's computer, or may be a web-based program on an array vendor's server that is accessed by a customer via the Internet, or a combination of both. Customer array selection is described below both in terms of using stand-alone programming 14 stored on customer computers 12a-12n, as well as through use of web-based programming 18 on vendor server 16.

At event 200, a customer or other potential array user selects one or more genes of interest for which arrays are to be designed. Gene selection will depend on the commercial or academic interests of the customer, and may be based on gene expression analysis goals, genomic characterization goals, or other goals or interests of the customer.

At event 210, programming 14 on client computer 12a, 12b or 12n is executed, or browser programming on client computers 12a-12n is executed together with web-based programming 18 on vendor server 16, to provide for customer selection of array design parameters as described in the events below. Programming 14 or 18 generates a visual user interface on the display (not shown) of client computer 12a-12n that allows the user thereof to select or specify parameters for use in the design of an array according to the gene selection made by the customer in event 200.

The displayed visual interface in this event may, for example, utilize “pull-down” menus to provide array parameter selection options or prompts to the user, “help” menus for providing instructions, graphical user interface (GUI) icons upon which a user may “click” with a mouse to make a selection, text fields in which a user may enter alphanumeric character strings using a keyboard, or other conventional visual interface tools. The design and use of visual interfaces of this sort is well known in the art. An exemplary visual interface that is used for nucleic acid microarray fabrication is provided by CLONETRACKER™ of BioDiscovery Inc., Los Angeles, Calif. Where web-based programming 18 is used to create a visual user interface on the display of client computers 12a-12n, the GUI and other visual interface tools may be based on Java applets embedded in HTML pages of programming 18 that are executed by browser programming stored on client computers 12a-12n.

At event 220, the visual interface provided by programming 14 or 18 presents the user with the choice of whether or not to select layout parameters for array design. The user choice may be provided, for example, as GUI icons that provide “yes?” or “no?” options or prompts for layout parameter selection. If yes, the customer elects to enter layout parameters, and event 230 is carried out. If no, the customer elects not to select any layout parameters, and event 300 may occur.

At event 230, the user enters or inputs selected layout parameters. The visual user interface generated by programming 14 or 18 allows the customer user to select or specify any or all layout parameters for designing an array. The visual user interface may include, for example, a gene specification (rather than a probe specification), for all non-control layout parameters, so that the customer may make layout parameter selections based on specified genes. Selections may be made, for example, by “clicking” on appropriate GUI or entering text descriptions of selectable layout parameters. Some exemplary selectable array layout parameters are shown in sub-events 240-270. At sub-event 240, the customer selects layout pattern parameters, which may include, for example, array size and shape considerations, and the number of features to be included in the array. At sub-event 250, the customer may select array density parameters in terms of the number of probe spots per substrate surface area. At sub-event 260, the customer may select layout parameters associated with the location of control probes (if any) to be included in the array. At sub-event 270, the customer may select layout parameters associated with the location of specific genes within the array. The array parameter selections provided in sub-events 240-270, it should be noted, are merely exemplary and do not define an inclusive list of selectable array layout parameters. Various additional selectable array layout parameters will suggest themselves to those skilled in the art, and may be selected in event 230.

Information associated with layout array parameter selection in event 230 may reside entirely within programming 14 or 18, or within a database stored in client computer 12a-12n. Alternatively, selectable layout array parameter information in databases 22a-22n may be used. Information in databases 22a-22n may be retrieved by database management software on vendor server 16 and presented by programming 18 to customers on client computers 12a-12n by visual interface in the form of HTML page-embedded Java applets. Databases 22a-22n may store layout parameter information based on previous layout parameter selections made by a customer for a gene of interest. The customer may retrieve these previously made layout parameter selections from databases 22a-22n and review then for possible use in the layout parameter selection of event 230.

At event 280, the visual interface provided by programming 14 or 18 generates a visual display of an array layout on the display of client computer 12a-12n, according to the layout parameters selected by the customer in event 230. The layout display allows the customer to view and evaluate the array layout design, and may allow the customer to visually adjust the displayed layout. Where web-based programming 18 is used, the layout display may be created by Java applets embedded in web pages of programming 18 and executed by a browser on client computer 12a-12n. Various ways of representing an array layout in a visual computer display are possible and may be used with the invention. The commercial software CLONETRACKER™ of BioDiscovery Inc., Los Angeles, Calif. provides an exemplary array layout display.

At event 290, programming 14 or 18 presents the customer with the option of revising the array layout parameters selected in event 230. This choice may be provided in the form of GUI “yes?” or “no?” icon options that the customer may select. If the customer elects yes to revise the layout parameters, event 230 is repeated, and the user may change or revise previously selected layout parameters, delete previously selected layout parameters, or select additional layout parameters not previously chosen. If the customer elects no, event 300 is carried out.

At event 300, the visual interface provided by programming 14 or 18 presents the user with the choice of whether or not to select probe-based parameters for array design. This choice may be embodied in displayed GUI icons that provide “yes?” or “no?” options for probe parameter selection. If the customer wishes to enter probe parameters and elects yes, event 310 is carried out. If the customer does not wish to select any probe parameters and elects no, event 370 occurs.

At event 310, the user enters or inputs selected probe parameters. The visual user interface generated by programming 14 or 18 may permit the customer user to select or specify any or all probe parameters needed for designing an array. In many situations, as noted above, customers will elect to defer selection of probe parameters associated with probe sequencing to an array vendor having the specialized software and experience needed for sequence curation and probe sequence determination. The visual user interface may provide for probe parameter selection via GUI icons as described above. Exemplary probe parameter selections are shown in sub-events 320-340. In sub-event 320, the customer may select probe length(s). Selectable probe lengths may comprise, for example, oligomeric nucleic acids (20-mer or less) of varying lengths, or “long-mer” nucleic acids having lengths greater than 20-mers. At sub-event 330, the customer may select the number of probes per gene in the array. At sub-event 340, the customer may select parameters associated with the use of replicate probes, such as the number of probes per gene versus the number of replicate probes.

Probe parameter selection information for event 310 may be contained within programming 14 or 18, within a database stored in client computer 12a-12n, within databases 22a-22n, or elsewhere. Databases 22a-22n may store probe parameter information based on previous probe parameter selections made by the customer, which can be retrieved from databases 22a-22n in event 310 for use in parameter selection. and review then for possible use in the layout parameter selection of event 230. Again, it should be noted that the probe parameter selections shown in sub-events 320-340 are only exemplary and not inclusive, and selection of other probe-related parameters may be carried out in event 310.

At event 350 the visual interface provided by programming 14 or 18 generates a visual display of an array layout on the display of client computer 12a-12n that shows the probe parameter selections made by the customer in event 310. The display of the selected probe parameters may be made in conjunction with, or independent of, the display of layout features according to layout parameters selected in event 230 according to the layout parameters selected by the customer in event 230. The display in event 320 allows the customer to view and evaluate the probe parameters selected.

At event 360, programming 14 or 18 presents the customer with the option of revising the probe parameters selected in event 310. This choice may be provided in the form of selectable GUI “yes?” or “no?”. A yes selection by the customer to revise the probe parameters at event 360 leads to repetition of event 230, wherein the customer may revise or delete previously selected probe parameters, or may select additional probe parameters not previously selected. A no selection in event 360 leads to event 370.

At event 370, programming 14 or 18 presents the user with the choice of selecting parameters associated with the use of control probes. GUI icons that provide “yes?” or “no?” options for control probe parameter selections may be provided by the visual interface generated by programming 14 or 18. If the customer wishes to enter control probe parameters and elects yes, event 380 is carried out. If the customer does not wish to select any probe parameters and elects no, event 440 occurs.

At event 380, the user may enter or input selectable control probe parameters according to prompting provided by programming 14 or 18 via the visual user interface displayed on client computer 12a-12n. Control probe parameter selection may be carried out using GUI icons in the visual display as described above. Possible selectable control probe parameters are shown in sub-events 390-410. At sub event 390, selection of the number of control probes for an array may be made by the customer. At sub-event 400, control probe sequence parameters are selected by the customer. Control probe sequence selection may be carried out by the customer in sub-event 400. In sub-event 410, the user may select parameters associated with the inclusion or exclusion of deletion control probes, insertion control probes, point mis-match control probes, or other types of control probes. Information for control probe selection may be integral to programming 14 or 18, be stored elsewhere within client computer 12a-12n, within databases 22a-22n, or elsewhere. Information associated with previous control probe parameter selections made by customers may be stored in databases 22a-22n for use in event 380.

The control probe parameter selections made in event 390 are displayed in event 420 by programming 14 or 18 via the visual interface generated and displayed on client computers 12a-12n. Display of control probe parameter selection may be provided together or concurrently with display of previously selected layout parameter and probe parameter selections made in events 230 and 310.

At event 430, the user may elect to revise the control probe selections of event 380 according to prompting by the visual interface provided by programming 14 or 18, The customer choice may be made through use of selectable GUI “yes?” or “no?” icons in the visual display. If the customer selects yes, event 380 is repeated, wherein the user may revise or delete control probe parameters, or select additional control probe parameters. A no selection results in event 440 being carried out.

At event 440, the selection of array design parameters by the customer is complete, and programming 14 or 18 creates one or more data files containing all of the array parameter selections made by the customer in events 230, 310 and 380. The customer-selected array parameter data files (not shown) may be outputted or otherwise transferred to a vendor by transmitting the data files via the Internet and vendor server 16 to databases 22a-22n, wherein the customer-selected array parameter data files are stored for use by the vendor for completion of the array design process. The data files may alternatively be transferred to the array vendor by recording the files onto a CD or other medium which is then mailed or delivered to the vendor.

Many variations on the method shown in FIG. 3 are possible, as will be readily apparent to persons skilled in the art. For reason of clarity, probe parameter selection has been shown in FIG. 3 in terms of layout parameter selection 230, probe parameter selection 310 and control probe parameter selection 380. Probe selection, however, may be organized in a variety of different manners however. Some or all of the aspects of the control probe parameter selection 380 could alternatively be considered as part of probe parameter selection 310. Similarly, various aspects of probe array selection 310, such as selection of the number of probes per gene, may alternatively be characterized as a layout parameter selection.

The method embodiment of FIG. 3 may also comprise one or more aspects of array parameter selection that are shown in FIG. 2 as part of the array parameters provided by a vendor. Sophisticated clients may wish to make parameter selections related to array fabrication, or may wish to be involved in selection of probes and probe sequence determination. It is also contemplated that, for particularly interesting genes, customers will pursue many array designs, and a substantial database of probe sequence information for these genes will be developed. Thus, databases 22a-22n or other databases may include a sufficient library of previously determined probes for specific gene such that the computational aspects of probe selection for that gene are no longer necessary. The array customer can make probe selections using programming 14 or 18 based on the stored, previously determined probe sequences. In other words, probe selection would involve a lookup process, rather than computation.

Computer Readable Media and Kits

The invention may provide one or more aspects of the above-described programming in the form of computer readable media having programming stored thereon, and kits which include the computer readable media. The computer readable media may be, for example, in the form a computer disk or CD, a floppy disk, a magnetic “hard card”, or any other computer readable media capable of containing program code stored electronically, magnetically, optically, or by other means. The computer readable media may comprise stored programming configured to, or otherwise capable of, allowing an array customer to input one or more selectable array parameters, and to generate a visual user interface which displays an array layout or other aspect(s) of an array design, as described above. The computer readable media may further comprise stored programming configured to allow an array vendor to utilize the array parameters selected by the customer for preparing a completed array design, and the programming may provide a visual user interface for parameter selection that permits selective inputting of parameters as well as selective deferring of parameter selections to a vendor, in the manner described above. The programming may be configured to allow customer parameter selection on a gene-specific basis.

The computer readable media may be present in kits usable for array design, which comprise computer readable media with the aforementioned programming stored thereon, together with printed instructions for the selection of array parameters by a customer, and for providing the selected array parameters to a vendor for selection of additional array parameters and completion of the array design. The kits may further comprise devices and materials for isolation and/or characterization of nucleic acids or other molecules of interest such as PCR (polymerase chain reaction) related items, as well as printed instructions associated with the isolation and/or characterization of the molecules of interest.

The array design systems, methods and kits of the invention are particularly well suited for gene-based array design wherein nucleic acid oligo probes are synthesized in situ on an array substrate surface. The number of such arrays required by a customer is usually relatively small, and probe design is critical to selection of a good probe. The invention may also be used in the design of probe-based arrays prepared by spotting techniques, including cDNA arrays which include clones of genes prepared in advance and wherein users simply “spot” the pre-prepared probes onto an array substrate with pin-based spotters. Such spotted cDNA arrays are perhaps the most common “custom” arrays currently available. The invention may additionally be used in design of spotted oligo arrays wherein oligonucleotide probes are synthesized in advance and applied to an array substrate by spotting.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for array design, comprising:
(a) selecting, by a customer, at least one array design parameter;
(b) providing said customer selected array design parameter to a vendor;
(c) providing, by said vendor, at least one additional array design parameter; and
(d) completing at least one array design according to said customer-selected array design parameters and said vendor provided array design parameters.

2. The method of claim 1, wherein said completing is carried out by said vendor.

3. The method of claim 1, wherein said completing is carried out by said customer.

4. The method of claim 1, wherein said array is a nucleic acid array, and said customer selected array design parameters are gene-based parameter selections.

5. The method of claim 4, wherein said customer selected array design parameters comprise layout parameters.

6. The method of claim 4, wherein said customer selected array design parameters comprise probe parameters.

7. The method of claim 4, wherein said customer selected array design parameters comprise control probe parameters.

8. The method of claim 1, further comprising generating a visual interface for said customer, said visual interface providing a display with parameter selection options for said selecting.

9. The method of claim 8, wherein said generating said visual interface further comprises generating a visual display of an array layout for said customer, based on said customer selected array design parameters.

10. The method of claim 9, further comprising reviewing, by said customer, said customer selected array design parameters, according to said visual display of said array layout.

11. The method of claim 9, further comprising revising, by said customer, said customer selected array design parameters.

12. A method for nucleic acid array design, comprising:
(a) selecting, by a customer, at least one gene-based array layout parameter;
(b) providing said customer selected gene-based array layout parameter to a vendor;
(c) providing, by said vendor, at least one probe selection parameter; and
(d) completing at least one array design according to said customer-selected array design parameters and said vendor provided array design parameters.

13. The method of claim 12, further comprising generating a visual interface for said customer on a customer computer, said visual interface providing a display with gene-based layout parameter selection options for said selecting.

14. The method of claim 13, wherein said generating said visual interface further comprises generating a visual display of an array layout for said customer, based on said customer selected gene-based array layout parameters.

15. The method of claim 14, further comprising reviewing, by said customer, said customer selected gene-based array layout parameter, according to said visual display of said array layout.

16. The method of claim 15, further comprising revising, by said customer, said customer selected gene-based array layout parameter.

17. A system for array design comprising at least one data processor, said data processor comprising;
(a) stored programming configured to allow an array customer to enter selectable array design parameters;
(b) stored programming configured to allow said array customer to view said selected array design parameters:
(c) stored programming configured to allow said array customer to revise said selected array design parameters; and
(d) stored programming configured to allow said array customer to output said selected array design parameters to an array vendor.

18. The system of claim 17, further comprising stored programming configured to generate a visual display for said array customer, said visual display configured to allow said array customer to enter said selectable array design parameters, view said selectable array design parameters, and revise said selectable array design parameters.

19. The system of claim 17, wherein said selectable array design parameters are gene-based array design parameters.

20. The system of claim 19, wherein said gene-based array design parameters are array layout parameters.

21. The system of claim 18, wherein said system comprises a vendor server computer, and at least one customer client computer operatively coupled thereto, said stored programming located on said vendor computer, said stored programming configured to generate said visual display on said customer client computer.

22. An array design system, comprising:
(a) means for selecting, by an array customer, at least one array design parameter;
(b) means for providing said customer selected array design parameter to a vendor;
(c) means for providing, by said vendor, at least one additional array design parameter; and
(d) means for completing at least one array design according to said customer-selected array design parameters and said vendor provided array design parameters.

23. A method for gene-based design of an in-situ array, comprising:
(a) selecting, by a customer, at least one gene of interest;
(b) selecting, by a customer, at least one array design parameter for said gene of interest;
(c) providing said customer selected array design parameter to a vendor;
(d) providing, by said vendor, at least one additional array design parameter for said gene of interest; and
(e) completing at least one array design according to said customer-selected array design parameters and said vendor provided array design parameters.

24. The method of claim 23, further comprising synthesizing nucleic acid probes on a substrate surface, according to said completed array design to provide said in-situ array.

25. The method of claim 23, wherein said array design parameter provided by said customer comprises a probe parameter.

26. The method of claim 23, wherein said array design parameter provided by said vendor comprises probe selection.

27. A method for gene-based array design, comprising:
(a) selecting, by a customer, at least one gene of interest;
(b) selecting, by said customer, at least one probe parameter for said gene of interest;
(c) selecting, by said customer, at least one array layout parameter for said gene of interest;
(d) curating, by a vendor, sequence information for said gene of interest; and
(e) selecting, by said vendor, a plurality of nucleic acid probes for said gene of interest.

28. The method of claim 27, further comprising synthesizing nucleic acid probes on a substrate surface, according to said completed array design to provide said in-situ array.

29. In a computer readable medium, stored programming for gene-based array design, comprising:
(a) programming configured to allow an array customer to input identification of at least one selected gene;
(b) programming configured to allow an array customer to input at least one selectable array parameter for said selected gene;
(c) programming configured to generate a visual user interface that displays an array layout based on said selectable array parameter for said selected gene; and
(d) programming configured to allow an array vendor to utilize said array parameter inputted by said customer to preparing a completed array design for said selected gene.

30. A kit for array gene-based array design by a customer comprising the computer readable media of claim 29, and printed instructions for inputting, by said customer, said selected gene and said selectable array parameter for said selected gene.
